(19) United States
(12) Patent Application Publication
(10) Pub. No.: US 2023/0347143 A1
(43) Pub. Date: Nov. 2, 2023

(54) METHOD OF FORMING CHLORIDE ION (C1-), HYPOCHLORITE ION (OC1-) AND HYPOCHLOROUS ACID (HOC1) IN THE HUMAN AND ANIMAL BODY

(71) Applicants: Serkan TUNC, Istanbul (TR); Sevil TUNC, Istanbul (TR)

(72) Inventors: Serkan TUNC, Istanbul (TR); Sevil TUNC, Istanbul (TR)

(73) Assignees: Serkan TUNC, Istanbul (TR); Sevil TUNC, Istanbul (TR)

(21) Appl. No.: 17/791,237

(22) PCT Filed: Aug. 16, 2021
(86) PCT No.: PCT/TR2021/050813

§ 371 Date: Jul. 7, 2022

(30) Foreign Application Priority Data
Sep. 17, 2020 (TR) 2020/14753

(51) Int. Cl. A61N 1/20

(52) U.S. Cl. CPC A61N 1/205

(57) ABSTRACT

A method of forming chloride ion (Cl⁻), hypochlorite ion (OCl⁻) and hypochlorous acid (HOCl) in human and animal bodies includes the steps of: decomposition of water (H₂O), as hydrogen (H⁺) and hydroxide (OH⁻) in tissue fluid and blood in the human or animal body; decomposition of sodium chloride (NaCl) as sodium (Na⁺) and chloride (Cl⁻); and decomposition of potassium chloride (KCl) as potassium (K⁺) and chloride (Cl⁻) with the energy created inside the human or animal body and thus eliminating the ion deficiency in the human and animal body. The method does not require adding any additional chemicals to the body, and uses the energy generated by passing the electric current through the body. The electric current is provided by the direct current electricity generator and the frequency generator that are connected to the body by serial connection.

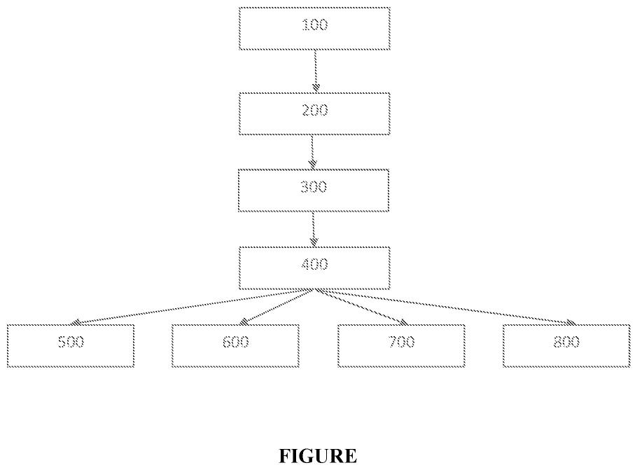

### CROSS REFERENCE TO THE RELATED APPLICATIONS

This application is the national phase entry of International Application No. PCT/TR2021/050813, filed on Aug. 16, 2021, which is based upon and claims priority to Turkish Patent Application No. 2020/14753, filed on Sep. 17, 2020, the entire contents of which are incorporated herein by reference.

### TECHNICAL FIELD

The invention is related to the separation of compounds in water (H₂O), sodium chloride (NaCl) and potassium chloride (KCl) salts in human and animal body tissue fluids and blood into hydrogen (H⁺), hydroxide (OH⁻), sodium (Na⁺), chloride (Cl⁻), potassium (K⁺) through the dissociation of molecular bonds and then formation of chloride ion (Cl⁻), hypochlorite ion (OCl⁻) and hypochlorous acid (HOCl) in human and animal bodies without adding any additional chemicals to the body, using the energy generated by passing the electric current through the body provided by the direct current electricity generator and the frequency generator, which the body is connected by serial connection.

### BACKGROUND

The chemical changes desired to be made in the human and animal body are initiated by the direct transmission of various chemicals to the body in the forms of solid, liquid and gas. To initiate a chemical reaction, the action of another chemical is used.

In the state of the art, when there is a lack of chloride ion (Cl⁻) in the human and animal body, this amount is increased by adding chemicals from the outside to the body.

In the state of the art, hypochlorite ion (OCl⁻) and hypochlorous acid (HOCl), which are used in the treatment of infectious diseases in human and animal bodies, are added to the body with various chemical compounds. In this case, undesirable side effects and results may be encountered.

In the state of the art, there is no method or treatment in the form of using the human and animal body as a resistance for electric current and thus dissociating the molecular bonds of chemical compounds.

### SUMMARY

The invention aims to connect the human and animal body in series with an electrical circuit to act as a resistor and to decompose the molecular bonds of water (H₂O), sodium chloride (NaCl) and potassium chloride (KCl) salts in tissue fluids and blood in human and animal bodies with the energy created on the human and animal body and thereby forming chloride ion (Cl⁻), hypochlorite ion (OCl⁻) and hypochlorous acid (HOCl) in tissue fluids and blood found in human and animal bodies.

### BRIEF DESCRIPTION OF THE FIGURES

The FIGURE for the method of forming chloride ion (Cl⁻), hypochlorite ion (OCl⁻) and hypochlorous acid (HOCl) in human and animal bodies is shown below.

FIGURE: Flow chart of the method of forming chloride ion (Cl⁻), hypochlorite ion (OCl⁻) and hypochlorous acid (HOCl) in human and animal bodies

### EXPLANATION OF THE REFERENCES IN THE FIGURE

The corresponding reference numbers shown on the flow chart for the method of forming chloride ion (Cl⁻), hypochlorite ion (OCl⁻) and hypochlorous acid (HOCl) in human and animal bodies are given below.
101. Generating electric current with direct current converted by a direct current generator from a circuit embodiment formed with a direct current electricity generator, an electrical frequency generator, a human or animal body that acts as a resistor, a switch that works on and off and coupling of said elements in series with each other with a conductor wire
201. Generating electrical frequency with electrical frequency generator and obtaining direct current with frequency that can be adjusted according to the desired chemical reaction
301. Ensuring the occurrence of chemical reactions in the tissue fluid and blood compounds in the human or animal body with the energy formed inside the human or animal body by passing the current through the human or animal body used as a resistor
401. Decomposition of water (H₂O), as hydrogen (H⁺) and hydroxide (OH⁻) in tissue fluid and blood in the human or animal body; decomposition of sodium chloride (NaCl) as sodium (Na⁺) and chloride (Cl⁻); decomposition of potassium chloride (KCl), as potassium (K⁺) and chloride (Cl⁻) with the energy created inside the human or animal body and thus eliminating the ion deficiency in the human and animal body
501. Ensuring that the chloride (Cl⁻) ions formed in the tissue fluid and blood in the human or animal body form a bond with the dissolved oxygen (O) in the same environment with the energy created inside the human or animal body and thereby forming hypochlorite (OCl⁻) ion
601. Ensuring that the hydrogen (H⁺) formed in the tissue fluid and blood in the human or animal body form a bond with hypochlorite (OCl⁻) with the energy created in the human or animal body and thereby forming hypochlorous acid (HOCl)
701. Ensuring that the sodium (Na⁺) and hydroxide (OH⁻) formed in the tissue fluid and blood in the human or animal body form a bond together with the energy created in the human or animal body, and thereby forming sodium hydroxide (NaOH)
801. Ensuring that the sodium (K⁺) and hydroxide (OH⁻) formed in the tissue fluid and blood in the human or animal body form a bond together with the energy created in the human or animal body, and thereby forming potassium hydroxide (KOH).

### DETAILED DESCRIPTION OF THE EMBODIMENTS

The invention relates to an electrical circuit created by making use of the ability of human and animal bodies to pass electric current and resist this current, and to create chemical reactions in tissue fluids and blood in human and animal bodies with the energy obtained thanks to this electric circuit.

The circuit in which the human and animal body is used to act as a resistor has an embodiment which is composed of a direct current generator that converts alternating current to direct current, an electrical frequency generator, a human/animal body that acts as a resistor, a switch operating on and off, and coupling of the said elements in series with each other with a conductive wire.

In the circuit created to achieve the aim of the invention, there is a direct current electricity generator that converts alternating current to direct current. In order to generate frequency in direct current, an electrical frequency generator has been integrated into the DC electricity generator by serial connection.

There is at least one switch that turns the circuit on and off in order to control the flow of current in the said circuit. The human or animal body is connected in series with the circuit as a resistor in order to generate a current in the circuit. In the circuit in question, in order to obtain higher efficiency from the human or animal body by using a lower electrical voltage, frequency generator that provides frequency characteristic to the current coming out of the direct current generator and is connected to the circuit in series, the direct current was added to the circuit by connecting to the output of the electricity generator.

When the energy level exceeding the molecular bond dissociation energy levels of water (H₂O), sodium chloride (NaCl) and potassium chloride (KCl) salts in the body is reached by giving current to the circuit in question, water (H₂O) decomposes into hydrogen (H⁺) and hydroxide (OH⁻); sodium chloride (NaCl) decomposes into sodium (Na⁺) and chloride (Cl⁻), and potassium chloride (KCl) decomposes into potassium (K⁺) and chloride (Cl⁻).

With the introduction of current into the circuit in question, it is ensured that the chloride (Cl⁻) ions bond with the oxygen (O) in the same environment and the hypochlorite (OCl⁻) ion is formed. With the introduction of current to the circuit, the hydrogen (H⁺) formed bonds with the hypochlorite (OCl⁻), resulting in the formation of hypochlorous acid (HOCl).

The invention is related to the separation of compounds in water (H₂O), sodium chloride (NaCl) and potassium chloride (KCl) salts in human and animal body tissue fluids and blood into hydrogen (H⁺), hydroxide (OH⁻), sodium (Na⁺), chloride (Cl⁻), potassium (K⁺) through the dissociation of molecular bonds and then formation of chloride ion (Cl⁻), hypochlorite ion (OCl⁻) and hypochlorous acid (HOCl) in human and animal bodies without adding any additional chemicals to the body, using the energy generated by passing the electric current through the body provided by the direct current electricity generator and the frequency generator, which the body is connected by serial connection.

The method of forming chloride ion (Cl⁻), hypochlorite ion (OCl⁻) and hypochlorous acid (HOCl) in human and animal bodies consists of the following steps:
generating electric current with direct current converted by a direct current generator from a circuit embodiment formed with a direct current electricity generator, an electrical frequency generator, a human or animal body that acts as a resistor, a switch that works on and off and coupling of said elements in series with each other with a conductor wire (100),
generating electrical frequency with electrical frequency generator and obtaining direct current with frequency that can be adjusted according to the desired chemical reaction (200),
ensuring the occurrence of chemical reactions in the tissue fluid and blood compounds in the human or animal body with the energy formed inside the human or animal body by passing the current through the human or animal body used as a resistor (300),
decomposition of water (H₂O), as hydrogen (H⁺) and hydroxide (OH⁻) in tissue fluid and blood in the human or animal body; decomposition of sodium chloride (NaCl) as sodium (Na⁺) and chloride (Cl⁻); decomposition of potassium chloride (KCl), as potassium (K⁺) and chloride (Cl⁻) with the energy created inside the human or animal body and thus eliminating the ion deficiency in the human and animal body (400),
ensuring that the chloride (Cl⁻) ions formed in the tissue fluid and blood in the human or animal body form a bond with the dissolved oxygen (O) in the same environment with the energy created inside the human or animal body and thereby forming hypochlorite (OCl⁻) ion (500),
ensuring that the hydrogen (H⁺) formed in the tissue fluid and blood in the human or animal body form a bond with hypochlorite (OCl⁻) with the energy created in the human or animal body and thereby forming hypochlorous acid (HOCl). (600),
ensuring that the sodium (Na⁺) and hydroxide (OH⁻) formed in the tissue fluid and blood in the human or animal body form a bond together with the energy created in the human or animal body, and thereby forming sodium hydroxide (NaOH) (700),
ensuring that the sodium (K⁺) and hydroxide (OH⁻) formed in the tissue fluid and blood in the human or animal body form a bond together with the energy created in the human or animal body, and thereby forming potassium hydroxide (KOH) (800).

### INDUSTRIAL APPLICATIONS OF THE INVENTION

The invention is a method used for forming various chemicals in tissue fluids and blood of human and animal body and is easily applicable to the industry.

What we claim is:

1. A method of forming chloride ion (Cl⁻), hypochlorite ion (OCl⁻), and hypochlorous acid (HOCl) in a human or animal body, comprising the following steps:
generating an electric current with direct current converted by a direct current generator from a circuit embodiment formed with elements comprising a direct current electricity generator, an electrical frequency generator, the human or animal body acting as a resistor, a switch for turning on and off and coupling of the elements with a conductor wire, wherein the elements are in series with each other,
generating electrical frequency with the electrical frequency generator and obtaining the direct current with a frequency, wherein the frequency is adjustable according to a desired chemical reaction,
ensuring an occurrence of the desired chemical reaction in tissue fluid and blood in the human or animal body with an energy formed inside the human or animal body by passing the electric current through the human or animal body,
decomposition of water (H₂O), as hydrogen (H⁺) and hydroxide (OH⁻) in the tissue fluid and the blood in the human or animal body; decomposition of sodium chloride (NaCl) as sodium (Na⁺) and chloride (Cl⁻); decomposition of potassium chloride (KCl), as potassium (K⁺) and chloride (Cl⁻) with the energy created inside the human or animal body and eliminating an ion deficiency in the human and animal body,
ensuring that the chloride (Cl⁻) ion formed in the tissue fluid and the blood in the human or animal body form a bond with dissolved oxygen (O₂) in a same environment with the energy created inside the human or animal body and thereby forming the hypochlorite (OCl⁻) ion,
ensuring that the hydrogen (H⁺) formed in the tissue fluid and the blood in the human or animal body form a bond with the hypochlorite (OCl⁻) ion with the energy created inside the human or animal body and thereby forming the hypochlorous acid (HOCl),
ensuring that the sodium (Na⁺) and hydroxide (OH⁻) formed in the tissue fluid and the blood in the human or animal body form a bond together with the energy created inside the human or animal body, and thereby forming sodium hydroxide (NaOH),
ensuring that the sodium (K⁺) and hydroxide (OH⁻) formed in the tissue fluid and the blood in the human or animal body form a bond together with the energy created inside the human or animal body, and thereby forming potassium hydroxide (KOH).
